(19) United States
(12) Patent Application Publication
(10) Pub. No.: US 2025/0233220 A1
(43) Pub. Date: Jul. 17, 2025

(54) Device and Method for Detecting a Defect in a Battery Arrangement for a Vehicle

(71) Applicant: Vitesco Technologies GmbH, Regensburg (DE)

(72) Inventors: Tobias Braun, Deuerling (DE); Philippe Grass, Regensburg (DE)

(73) Assignee: Vitesco Technologies GmbH, Regensburg (DE)

(21) Appl. No.: 18/852,990

(22) PCT Filed: Apr. 5, 2023
(86) PCT No.: PCT/EP2023/058998

§ 371 Date: Sep. 30, 2024

(30) Foreign Application Priority Data
Apr. 12, 2022 (DE) 10 2022 203 664.6

(51) Int. Cl. H01M 10/48; G01N 33/00; H01M 50/30; H01M 50/342

(52) U.S. Cl. CPC H01M 10/488; G01N 33/0063; H01M 50/3425; H01M 50/394

(57) ABSTRACT

Various embodiments of the teachings herein include a device for detecting a defect of an arrangement including a battery housing and a battery cell. An example includes: a first sensor for a gas component within the battery housing; a second gas for the at least one gas component outside the battery housing; and a control apparatus receiving the signals. The control apparatus identifies a defect in the arrangement if the presence and/or a content of the gas component within the battery housing exceeds a predetermined first content threshold value and the absence and/or a content of the gas component outside the battery housing falls below a predetermined second content threshold value.

### CROSS-REFERENCE TO RELATED APPLICATIONS

This application is a U.S. National Stage Application of International Application No. PCT/EP2023/058998 filed Apr. 5, 2023, which designates the United States of America, and claims priority to DE Application No. 10 2022 203 664.6 filed Apr. 12, 2012, the contents of which are hereby incorporated by reference in their entirety.

### TECHNICAL FIELD

The present disclosure relates to storage of electrical energy. Various embodiments of the teachings herein include systems and/or methods for detecting a defect in a battery arrangement for a vehicle.

### BACKGROUND

Lithium-ion batteries are currently used in electromobility, both for hybrid vehicles and for all-electric vehicles. In the case of lithium-ion batteries, aluminum electrodes on the cathode side and copper electrodes on the anode side are used as current collectors, each usually in film form. Lithium transition metal oxides, such as cobalt, manganese, and nickel, for example, find application as cathode material and carbon/graphite find application as anode material. The electrolyte situated therebetween consists of organic solvent with dissolved electrolyte salts. In order to avoid short circuits, a lithium ion-permeable separator (e.g. composed of polypropylene) may be positioned between the electrodes.

Key for long-lasting operation is both that the battery cells are not overcharged or discharged too much, since faster aging with deactivation of active constituents of the electrodes and an increase in the cell impedance and also thermal “runaway” of the respective cell, i.e. total failure, may occur. For this purpose, it is of crucial importance to know the present state of charge.

During the operation of lithium-ion batteries, it is important to operate the battery in the correct temperature range. Battery overheating, in particular, is deemed to be particularly hazardous since unstoppable thermal runaway may occur starting from a critical temperature. In that case, the individual components of the battery cell react with one another in an uncontrolled manner. With very great evolution of heat and gas formation, the cell reacts until all the components have fully reacted.

During thermal runaway, gas formation occurs in the respective battery cell and the internal pressure increases until the housing of the battery cell yields and the latter ruptures. The gas generated thus escapes. The battery cell is heated up further until the uncontrollable reaction starts and the battery cell abruptly reacts completely. The gases produced and released in the process can include hydrogen, carbon dioxide, carbon monoxide and hydrocarbons such as methane or ethane. Depending on the progress of the reaction and thus of thermal runaway, characteristic gas concentrations can be measurable, whereby the state of the battery can be deduced.

Consequently, the information that there is a certain gas inside the battery housing is important on the one hand to recognize whether there is an ignitable gas mixture inside the battery housing, and on the other hand to detect a thermal runaway of at least one of the battery cells at an early stage. In addition, slow (cold) degassing could provide additional information about the condition of the battery cells. In conjunction with other sensors of the battery management system (voltage/current sensors), a more precise diagnosis of the condition of the battery or individual cells could thus be made.

Future monitoring systems will therefore use one or more different gas sensors that are energized continuously or at significant intervals and can therefore detect the gas concentrations inside the battery housing. The gas sensor can thus permanently or cyclically detect the composition of the gas or gas mixture present within the battery housing and output a gas signal on the basis of which the state of the battery can be assessed.

Today's battery housings usually have a pressure equalization valve to at least partially compensate for the influence of different ambient pressures and/or temperatures on the pressure inside the battery housing. Any gases and gas mixtures can flow out of the battery housing and into the battery housing via the pressure equalization valve. This means that a gas sensor located inside the battery housing would also generate a gas signal if a gas component that can be detected by the gas sensor flows into the battery housing from outside. When analyzing the gas signal from the gas sensor located inside the battery housing, a defect in the battery arrangement would be falsely indicated in such a case, such as outgassing of one of the battery cells or the start of a thermal runaway.

### SUMMARY

The present disclosure describes systems and methods useful for identifying a defect in a battery arrangement for a vehicle in a simple, favorable and reliable manner. For example, some embodiments of the teachings herein include a device (200) for detecting a defect of a battery arrangement (100), comprising a battery housing (110) and at least one battery cell (120) arranged in the battery housing (110), wherein the device (200) has: a first gas sensor (210) designed to be sensitive to at least one gas component present within the battery housing (110) and to generate a first gas signal indicating the presence and/or the content of the gas component within the battery housing (110), a second gas sensor (220) designed to be sensitive to the at least one gas component present outside the battery housing (110) and to generate a second gas signal indicating the presence and/or the content of the gas component outside the battery housing (110), and a control apparatus (230) which is connected to the first gas sensor (210) and the second gas sensor (220) and is designed to receive the first gas signal and second gas signal, wherein the control apparatus (230) is further designed to detect a defect in the battery arrangement (100) when the first gas signal indicates the presence and/or a content of the gas component within the battery housing (110) that exceeds a predetermined first content threshold value and the second gas signal indicates the absence and/or a content of the gas component outside the battery housing (110) that falls below a predetermined second content threshold value.

In some embodiments, there is a device housing (140) in which the first gas sensor (210) and second gas sensor (220) are at least partially arranged and which is designed to be attached to the battery housing (110) in such a way that an opening (112) provided in the battery housing (110) is at least partially sealed airtight by the device housing (240).

In some embodiments, there is a printed circuit board (244), which is arranged in the device housing (240) and to which the first gas sensor (210) and the second gas sensor (220) are electrically connected, wherein the printed circuit board (244) is further electrically connected to the control device (230).

In some embodiments, the first gas sensor (210) extends sealingly from the interior of the device housing (240) at least partially into the interior of the battery housing (110), and/or the second gas sensor (220) extends sealingly from the interior of the device housing (240) into the environment of the device housing (240) and the battery housing (110).

In some embodiments, there is a pressure equalization device (250) which is designed to at least partially equalize a pressure difference between the inside of the battery housing (110) and the outside of the battery housing (110) and to aerate or deaerate the battery housing (110).

In some embodiments, the pressure equalization device (250) is arranged on the device housing (240) in such a way that the fluid flowing out of the battery housing (110) or flowing into the battery housing (110) through the pressure equalization device (250) flows at least partially past the second gas sensor (220).

In some embodiments, there is an emergency deaeration device (260) which is designed to deaerate the battery housing (110) when the pressure inside the battery housing (110) exceeds a predetermined pressure threshold value.

In some embodiments, the emergency deaeration device (260) is arranged on the device housing (240) in such a way that the fluid flowing out of the battery housing (110) through the emergency deaeration device (260) flows at least partially past the second gas sensor (220).

In some embodiments, there is a flow guiding device (241) which is attached to the device housing (240) and extends at least partially into the interior of the battery housing (110) in such a way that the fluid flowing out of the battery housing (110) through the pressure equalization device (250) is at least partially guided past the first gas sensor (220).

In some embodiments, there is a device flange (242), which is attached to the device housing (240) and by means of which the device housing (240) can be fastened to the battery housing (110).

In some embodiments, there is the device housing (240) has a housing region (246), which is fluidically connected to the interior of the battery housing (110), and a further housing region (248), which is fluidically connected to the exterior of the battery housing (110) and separated from the housing region (246) by means of a housing wall (243).

As another example, some embodiments include a battery arrangement (100) for a vehicle, comprising: a battery housing (110) with at least one battery cell (120) arranged therein, and a device (200) as described herein.

As another example, some embodiments include a method for detecting a defect of a battery arrangement (100) comprising a battery housing (110) and at least one battery cell (120) arranged in the battery housing (110), wherein there are provided a first gas sensor (210) which is designed to be sensitive to at least one gas component present inside the battery housing (110) and to generate a first gas signal which indicates the presence and/or the content of the gas component inside the battery housing (110), and a second gas sensor (220) which is designed to be sensitive to the at least one gas component present outside the battery housing (110) and to generate a second gas signal indicating the presence and/or content of the gas component outside the battery housing (110), wherein the method comprises: generating a first gas signal by means of the first gas sensor (210), generating a second gas signal by means of the second gas sensor (220), and detecting a defect in the battery arrangement (100) if the first gas signal indicates the presence and/or a content of the gas component inside the battery housing (110) that exceeds a predetermined first content threshold value, and the second gas signal indicates the absence and/or a content of the gas component outside the battery housing (110) that falls below a predetermined second content threshold value.

In some embodiments, the second gas signal is generated after a predetermined period of time has elapsed after the first gas signal has been generated, and the method further comprises: detecting a defect in the battery arrangement (100) if the first gas signal indicates the presence and/or a content of the gas component inside the battery housing (110) that exceeds a predetermined first content threshold value, and the second gas signal indicates the presence and/or a content of the gas component outside the battery housing (110) that falls below a predetermined third content threshold value.

In some embodiments, the method further comprises detecting a defect of the first gas sensor (210) and/or second gas sensor (220) if only one of the two gas sensors (210, 220) generates a gas signal indicating the presence and/or a content of the gas component which is greater than the predetermined first content threshold value.

### BRIEF DESCRIPTION OF THE DRAWINGS

Further advantages and features of the teachings of the present disclosure are apparent to a person skilled in the art by putting the teaching described herein into practice and taking into consideration the accompanying single drawing, in which:

FIG. 1 shows a schematic illustration of a device incorporating teachings of the present disclosure for a battery arrangement for a vehicle;

FIG. 2 shows a schematic illustration of a further device incorporating teachings of the present disclosure for a battery arrangement for a vehicle;

FIG. 3 shows a schematic illustration of a further device incorporating teachings of the present disclosure for a battery arrangement for a vehicle;

FIG. 4 shows a schematic illustration of a further device incorporating teachings of the present disclosure for a battery arrangement for a vehicle;

FIG. 5 shows a schematic illustration of a further device incorporating teachings of the present disclosure for a battery arrangement for a vehicle; and

FIG. 6 shows a flow diagram of an example method incorporating teachings of the present disclosure for detecting a defect of the battery arrangement of FIG. 4.

### DETAILED DESCRIPTION

The teachings of the present disclosure include systems and methods that equip a battery arrangement with a device that has two gas sensors. Each of the two gas sensors is designed to detect the presence and/or the content of at least one gas component, wherein one of the two gas sensors detects the presence and/or the content of the gas component inside the battery housing of the battery arrangement and the other of the two gas sensors detects the presence and/or the content of the gas component outside the battery housing. This allows the gas signal from the gas sensor located inside the battery housing to be checked for plausibility with the gas signal from the gas sensor located outside the battery housing. This means, for example, that a warning can be issued with greater certainty that there is a defect in the battery arrangement, such as thermal runaway of one of the battery cells in the battery arrangement.

The battery housing of battery arrangements is usually provided with pressure equalization devices, by means of which pressure equalization and thus a fluid flow between the outside and the inside of the battery housing is made possible. Therefore, if the gas component flows into the battery housing from outside the battery housing, this can be detected by a signal from each of the two gas sensors, which means that no warning can be issued. In particular, a warning to the driver of the vehicle can thus be avoided, as despite the gas signal from the gas sensor located inside the battery housing, no warning is issued, as the detection of the presence of the gas component outside the battery housing makes it possible to determine that the gas components do not originate from a defect in the battery arrangement, but rather have flowed in from outside.

Consequently, some embodiments of the teachings herein include a device for detecting a defect of a battery arrangement comprising a battery housing and at least one battery cell arranged in the battery housing. The device has a first gas sensor sensitive to at least one gas component present inside the battery housing and generating a first gas signal which indicates the presence and/or the content of the gas component inside the battery housing, a second gas sensor sensitive to the at least one gas component present outside the battery housing and generating a second gas signal indicating the presence and/or content of the gas component outside the battery housing, and a control apparatus connected to the first gas sensor and the second gas sensor and designed to receive the first gas signal and the second gas signal.

The control apparatus is also designed to detect a defect in the battery arrangement if the first gas signal indicates the presence and/or a content of the gas component inside the battery housing that exceeds a predetermined first content threshold value, and the second gas signal indicates the absence and/or a content of the gas component outside the battery housing that falls below a predetermined second content threshold value. The gas signal of the first gas sensor is thus checked for plausibility with the gas signal of the second gas sensor when a gas component is detected inside the battery housing. If the determined gas component is also present outside the battery housing within a very short time before the presence of the gas component inside the battery housing is detected, it can be assumed that the origin of the gas component is outside the battery housing and consequently that there is no defect in the battery arrangement, in particular in the at least one battery cell. Instead, it can be assumed that the gas component detected inside the battery arrangement with the first gas sensor has flowed into the battery housing from outside.

In some embodiments, there is a device housing in which the first gas sensor and the second gas least partially arranged and which is designed to be attached to the battery housing in such a way that an opening provided in the battery housing is at least partially sealed airtight by the device housing. The device housing can form part of the battery housing and the first gas sensor and second gas sensor can be arranged relatively in such a way that the first gas sensor detects the gas component inside the battery housing and the second gas sensor detects the gas component outside the battery housing.

In some embodiments, there is a printed circuit board, which is arranged in the device housing and to which the first gas sensor and the second gas sensor are electrically connected. The printed circuit board is also electrically connected to the control apparatus, preferably via a suitable connector arrangement.

In some embodiments, the first gas sensor extends sealingly from the interior of the device housing at least partially into the interior of the battery housing, and/or if the second gas sensor extends sealingly from the interior of the device housing into the environment of the device housing and the battery housing. In particular, this ensures that the first gas sensor detects the gas component inside the battery housing and the second gas sensor detects the gas component outside the battery housing.

In some embodiments, there is a pressure equalization device which is designed to at least partially equalize a pressure difference between the inside of the battery housing and the outside of the battery housing and to aerate or deaerate the battery housing. In particular, a fluid flow, for example the ambient air, can flow into the battery housing and flow out of the battery housing through the pressure equalization device. The device can thus additionally have the function of a pressure equalization device and provide the battery housing with aeration or deaeration. Consequently, an at least partially compact device can be created which can provide the first gas sensor, the second gas sensor and the pressure equalization device for the battery arrangement.

In some embodiments, the pressure equalization device is arranged on the device housing in such a way that the fluid flowing out of the battery housing or the fluid flowing into the battery housing flows at least partially past the second gas sensor and can be measured by it.

In some embodiments, there is an emergency deaeration device which is designed to deaerate the battery housing when the pressure inside the battery housing exceeds a predetermined pressure threshold value. The emergency deaeration device can be designed as a so-called rupture disk, for example, which is arranged in an opening in the device housing. If the predetermined pressure threshold value is exceeded inside the battery housing, the rupture disk breaks, allowing the gas mixture that has accumulated inside the battery housing to flow efficiently and rapidly out of the interior of the battery housing through the opening that is then present. This can prevent the battery housing from exploding, for example.

In some embodiments, the emergency deaeration device is arranged on the device housing in such a way that the fluid flowing out of the battery housing through the emergency deaeration device flows at least partially past the second gas sensor. This in turn can ensure that the fluid flowing out of the battery housing through the emergency deaeration device can be measured by the second gas sensor.

In some embodiments, there is a flow guiding device which is attached to the device housing and extends at least partially into the interior of the battery housing in such a way that the fluid flowing out of the battery housing through the pressure equalization device and/or emergency deaeration device is at least partially guided past the first gas sensor. The flow guiding device can therefore in turn ensure that the fluid present in the battery housing cannot flow out of the battery housing undetected by the first gas sensor. If, for example, there is a defect in the battery arrangement, such as the at least one battery cell, the first gas sensor can detect the gas component that may be present with a high degree of certainty.

In some embodiments, there is a device flange attached to the device housing and by means of which the device housing can be fastened to the battery housing. The flange can, for example, be constituted by projections projecting outwards from the battery housing which, after insertion of the device housing into the opening provided in the battery housing, are supported on the outer wall of the battery housing and provide a connection option of the battery housing.

In some embodiments, the device housing has a first housing region, which is fluidically connected to the interior of the battery housing, and a second housing region, which is fluidically connected to the exterior of the battery housing and separated from the first housing region by means of a housing wall.

Some embodiments include a battery arrangement for a vehicle, which has a battery housing in which at least one battery cell is arranged, and one or more of the devices described herein.

Some embodiments include a method for detecting a defect of a battery arrangement comprising a battery housing and at least one battery cell arranged in the battery housing. Further provided are a first gas sensor which is designed to be sensitive to at least one gas component present inside the battery housing and to generate a first gas signal which indicates the presence and/or the content of the gas component inside the battery housing, and a second gas sensor which is designed to be sensitive to the at least one gas component present outside the battery housing and to generate a second gas signal indicating the presence and/or content of the gas component outside the battery housing. The method comprises generation of a first gas signal by means of the first gas sensor, generation of a second gas signal by means of a second gas sensor, and detection of a defect of the battery arrangement if the first gas signal indicates the presence and/or a content of the gas component inside the battery housing that exceeds a predetermined first content threshold value, and the second gas signal indicates the absence and/or a content of the gas component outside the battery housing that falls below a predetermined second content threshold value.

The methods described herein can thus be used to reliably detect a defect in the battery arrangement, as the first gas signal can be checked for plausibility with the second gas signal. This can be used, for example, to detect a faulty defect in the battery arrangement if, for example, the gas component flows into the battery housing from outside the battery housing and consequently there is no defect in the battery arrangement, such as one of the battery cells.

In some embodiments, the second gas signal is generated after a predetermined period of time has elapsed after the first gas signal has been generated. The method then further comprises detection of a defect in the battery arrangement if the first gas signal indicates the presence and/or a content of the gas component inside the battery housing that exceeds the predetermined first content threshold value, and the second gas signal indicates the presence and/or a content of the gas component outside the battery housing that exceeds a predetermined third content threshold value.

Therefore, if the chronological order of the detection of the gas component is such that the gas component inside the battery housing is detected first and the gas component outside the battery arrangement is also detected at a later time, it can be assumed that the origin of the gas component is inside the battery housing and consequently a defect in the battery arrangement can be detected.

In some embodiments, the method further comprises detecting a defect of the first gas sensor and/or second gas sensor if only one of the two gas sensors generates a gas signal indicating the presence of a content of the gas component which is greater than the predetermined first content threshold value. Use can be made of the fact that in the presence of the gas component, whether inside or outside the battery housing, both gas sensors would have to detect the gas component sooner or later due to a pressure equalization device of the battery arrangement. However, if this is not the case, it can be determined that at least one of the two gas sensors is defective.

In the context of the present disclosure, a gas signal describes an electrical signal of a gas sensor which can, on the one hand, qualitatively indicate the presence of one or more predetermined gas components, such as hydrogen, carbon dioxide, carbon monoxide and hydrocarbons, such as methane or ethane, and can, on the other hand, quantitatively indicate the content of the one or more gas components.

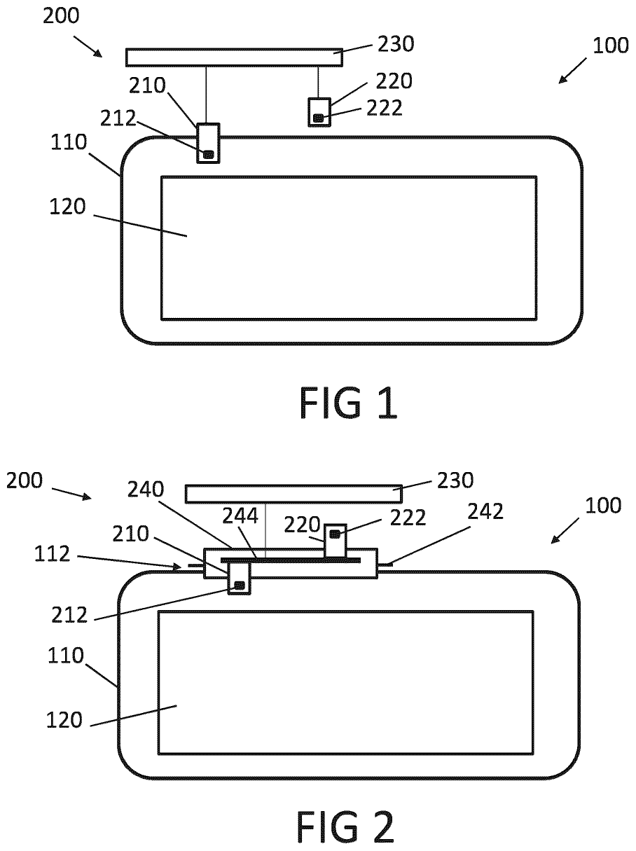

FIG. 1 shows a battery arrangement 100 incorporating teachings of the present disclosure, comprising a battery housing 110, designed to accommodate at least one battery cell 120, and a device 200 incorporating teachings of the present disclosure. The at least one battery cell 120 is schematically illustrated as a block in FIG. 1, wherein it is self-evident to a person skilled in the art that the at least one battery cell can be arranged.

The device 200 has a first gas sensor 210, which is designed to be sensitive to at least one gas component present within the battery housing 110 and to generate a first gas signal indicating the presence and/or the content of the gas component within the battery housing 110. The first gas sensor 210 can thus both qualitatively and quantitatively indicate the content of the gas component within the battery housing 110.

In some embodiments, the first gas sensor 210 is designed to be sensitive to such gas components which are generated during abnormal chemical reactions within the battery housing 110. In particular, the first gas sensor 210 can detect such an abnormal chemical reaction. By way of example, during thermal runaway of the at least one battery cell 120, hydrogen, carbon dioxide, carbon monoxide and hydrocarbons, such as methane or ethane, for example, may be generated and can be detected by the first gas sensor 210. Depending on the progress of the reaction and thus of thermal runaway, the first gas sensor 210 can detect the characteristic gas concentrations of the respective gas component, whereby the state of the battery or battery cells 120 can be deduced.

The device 200 further has a second gas sensor 220, which is designed to be sensitive to the at least one gas component present outside the battery housing 110 and to generate a second gas signal indicating the presence and/or the content of the gas component outside the battery housing 110. The second gas sensor 220 has a configuration similar to the configuration of the first gas sensor 210 and can be sensitive to the same gas components as the first gas sensor 210.

The first gas sensor 210 has a sensor element 212 that is sensitive to the gas component. The second gas sensor 220 likewise has a sensor element 222 that is sensitive to the gas component. As shown in FIG. 1, the first gas sensor 210 can, for example, be inserted into the battery housing 110 such that the sensor element 212 of the first gas sensor 210 is located at least partially within the battery housing 110. In contrast, the second gas sensor 220 is arranged such that the second sensor element 222 of the second gas sensor 220 is located at least partially outside the battery housing 110.

The device 200 of FIG. 1 also has a control apparatus 230, which is electrically connected to the first gas sensor 210 and the second gas sensor 220. The control apparatus 230 can receive and process the first gas signal from the first gas sensor 210 and the second gas signal from the second gas sensor 220. In particular, the control apparatus 230 can detect a defect in the battery arrangement 100, in particular outgassing or thermal runaway of the at least one battery cell 120. If the first gas signal indicates the presence and/or a content of the gas component inside the battery housing 110 that exceeds a predetermined first content threshold value, but the second gas signal indicates the absence and/or a content of the gas component outside the battery housing 110 that falls below a predetermined second content threshold value, a defect of the battery arrangement 100 may be detected. In particular, it can be assumed that the gas component has its origin inside the battery housing 110, since the first gas sensor 210 detects the gas component, but the second gas sensor 220 does not (yet). In such a case, a warning can be issued to the driver of the vehicle.

FIG. 2 shows a schematic illustration of a further device 200 incorporating teachings of the present disclosure for a battery arrangement 100 for a vehicle. The device 200 of FIG. 2 is similar to the device 200 of FIG. 1 and further comprises, in addition to the first gas sensor 210 and the second gas sensor 220, a device housing 240 in which the first gas sensor 210 and the second gas sensor 220 are at least partially arranged. The device housing 240 is configured to be attached to the battery housing 110 such that an opening 112 provided in the battery housing 110 is at least partially sealed airtight by the device housing 240. According to the device 200 of FIG. 2, the device housing 240 may have a device flange 242 for this purpose, which extends substantially outwardly from the device housing 240 and is supported on an outer wall of the battery housing 110 and provides a connection option between the device housing 240 and the battery housing 110. For example, the device housing 240 may be welded or soldered to the battery housing 110 at the device flange 242.

Within the battery housing 240 there is provided a printed circuit board 244, to which both the first gas sensor 210 and the second gas sensor 220 are connected. The printed circuit board 244, in turn, is electrically connected to the control apparatus 230 in such a way that the first gas signals generated by the first gas sensor 210 and the second gas signals generated by the second gas sensor 220 can be received and processed by the control apparatus 230.

The device 200 of FIG. 2 thus presents a compact unit consisting of a device housing 240 and the gas sensors 210, 220 arranged at least partially therein, which can be attached relatively easily to the battery housing 100. In particular, the device housing 240 forms a part of the battery housing 110 by being inserted into the opening 212 of the battery housing 110.

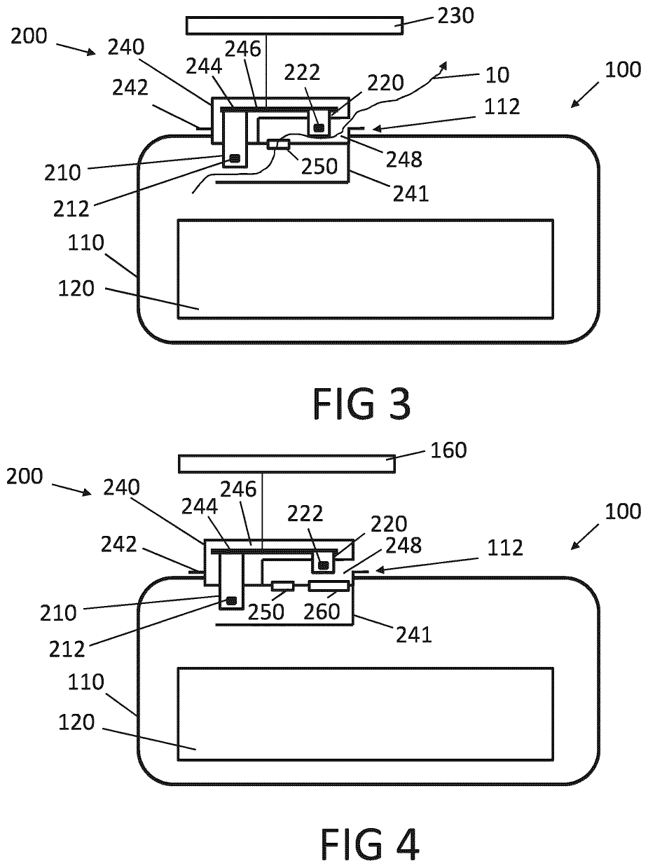

FIG. 3 shows a further schematic representation of a device 200 incorporating teachings of the present disclosure for a battery arrangement 100 for a vehicle. The device 200 of FIG. 3 is similar to the device 200 of FIG. 2 and differs in that the device housing 240 has, in addition to a first housing region 246 in which the printed circuit board 244 is arranged, a second housing region 248 which is separated from the first housing region 246. In particular, the second housing region 248 is arranged such that a flow path from the interior of the battery housing 110 to the exterior or the surroundings of the battery housing 110 is made possible by the second housing region 248 (see exemplary flow path in FIG. 3, which is indicated by the arrow 10).

The second gas sensor 220 is arranged such that the second sensor element 222 of the second gas sensor 220 is located at least partially within the second housing region 248.

The device 200 of FIG. 3 also has a pressure equalization device 250, which is attached to the device housing 240 and which is capable of providing a fluidic connection between the interior of the battery housing 110 and the second housing region 248 for equalizing the pressure between the exterior of the battery housing 110 and the interior of the battery housing 110. Consequently, the fluid flow path 10 of FIG. 3 extends through the pressure equalization opening 250. It is self-evident to a person skilled in the art that the direction of the fluid flow path 10 can also be in the opposite direction, namely from the exterior of the battery housing 110 through the second housing region 248 and the pressure equalization device 250 into the interior of the battery housing 210.

The device 200 of FIG. 3 further comprises a flow guiding device 241 extending at least partially into the interior of the battery housing 110 such that the fluid flowing from the battery housing 110 through the device 200, in particular through the second housing region 248, is at least partially guided past the first gas sensor 210. This can ensure that in the event of a defect in the battery arrangement 100, in particular the at least one battery cell 120, the gas generated in the process with the gas component sensitive to the gas sensor 210 flows at least partially past the sensor element 212 of the first gas sensor 210 and can be detected by it.

The device 200 of FIG. 3 can thus be used to create a compact unit consisting of the device housing 200, first gas sensor 210, second gas sensor 220 and pressure equalization device 250, which can be attached to the battery housing 110 as a compact unit.

FIG. 4 shows a further schematic illustration of a further embodiment of a device 200 incorporating teachings of the present disclosure for a battery arrangement 100 for a vehicle. The device 200 of FIG. 4 is substantially the same as the device 200 of FIG. 3 and further comprises an emergency deaeration device 260 which, similarly to the pressure equalization device 250, may allow a fluidic connection between the interior of the battery housing 110 and the second housing region 248, and thus the exterior of the battery housing 110, in the event of an emergency. For example, the emergency deaeration device 260 may be a rupture disk that can establish the aforementioned fluidic connection when the pressure inside the battery housing 110 exceeds a predetermined pressure threshold value, for example approximately 50 mbar above ambient pressure. In such a case, the rupture disk can break and thus completely deaerate the battery housing 110.

The device 200 of FIG. 4 can thus be used to create a compact unit consisting of the device housing 200, first gas sensor 210, second gas sensor 220, pressure equalization device 250 and emergency deaeration device 260, which can be attached to the battery housing 110 as a compact unit. In particular, the emergency deaeration device 260 is configured to increase the pressure equalization capacity of the pressure equalization device 250 when the predetermined pressure threshold value inside the battery housing 110 is exceeded. This means that the excess pressure inside the battery housing 110 can be equalized more quickly.

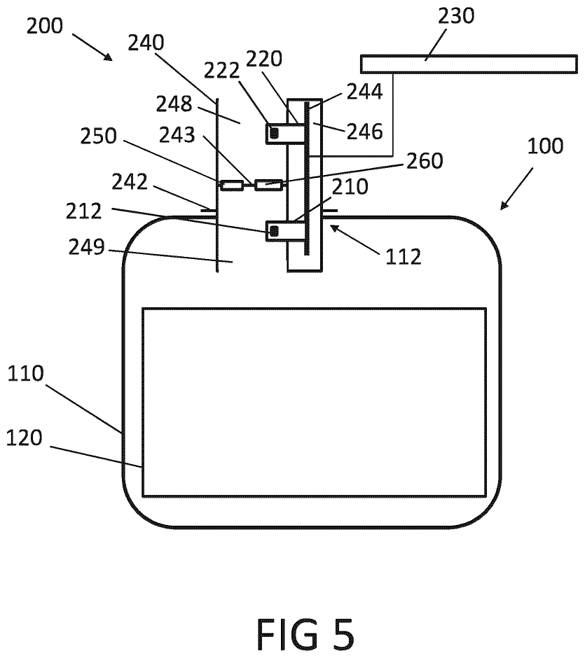

FIG. 5 shows yet another device 200 incorporating teachings of the present disclosure for a battery arrangement 100 for a vehicle. The device 200 of FIG. 5 further comprises a third housing region 249, which is fluidically connected to the interior of the battery housing 110 and within which the sensor element 212 of the first gas sensor 210 can detect the gas component. The third housing region 249 is separated from the second housing region 248, within which the sensor element 222 of the second gas sensor 220 can detect the gas component, by means of a housing wall 243, wherein the pressure equalization device 250 and the emergency deaeration device 260 can be arranged in the housing wall 243, which can establish a fluidic connection between the third housing region 249 and the second housing region 248. In the embodiment of the device 200 of FIG. 5, the device housing 240 can also be attached to the battery housing 110 via the device flange 240.

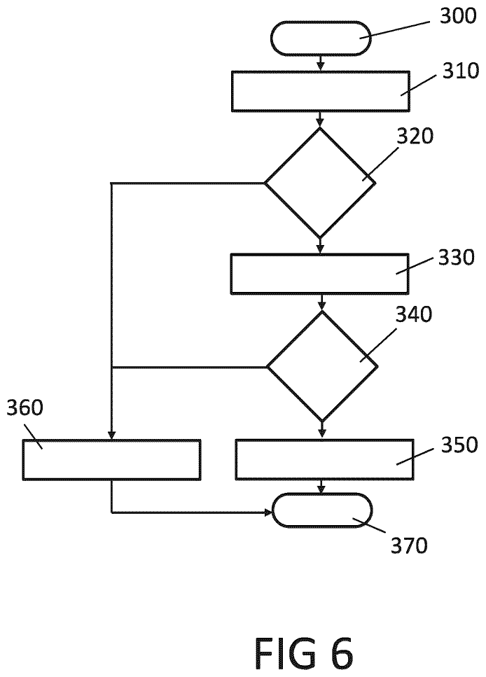

FIG. 6 shows an exemplary flow diagram of a method incorporating teachings of the present disclosure for detecting a defect of the battery arrangement 100, by way of example by means of the device 200 of FIG. 4. However, the method shown in FIG. 6 can also be carried out with the devices 200 of FIGS. 1 to 3 and FIG. 5.

The method of FIG. 6 starts at step 300 and then proceeds to step 320, in which a first gas signal is generated by means of the first gas sensor 210. In a subsequent step 330, a check is made to determine whether the first gas signal indicates the presence and/or a content of the gas component within the battery housing 110 that exceeds a predetermined first content threshold value. If it is determined in step 320 that the gas signal indicates the presence and/or a content of the gas component within the battery housing 110 that does not exceed the predetermined first content threshold value, the method proceeds to step 360 where a correct battery arrangement 110 is determined before the method ends at step 370.

However, if it is determined in step 320 that the first gas signal indicates the presence and/or a content of the gas component within the battery housing 110 that exceeds the predetermined first content threshold value, the method proceeds to step 330 at which a second gas signal is generated by means of the second gas sensor 220. The second gas signal is evaluated in step 340. If it is determined in step 340 that the second gas signal indicates the presence and/or a content of the gas component outside the battery housing 110 that exceeds a predetermined second content threshold value, the method returns to step 360, at which no defective battery arrangement 100 is detected, before the method again ends at step 370.

In this case, it can be assumed that the gas component is present both outside and inside the battery housing 110. In particular, the gas component was most likely first present outside the battery housing 110 and flowed into the interior of the battery housing 110 via the pressure equalization opening, for example. The generation of the first and second gas signals in steps 310 and 330 may take place simultaneously.

However, if it is determined in step 340 that the second gas signal indicates an absence and/or a content of the gas component outside the battery housing 110 that falls below the predetermined second content threshold value, the method proceeds to step 350, at which a defect in the battery arrangement 100, for example the at least one battery cell 120, is detected before the method again ends at step 370. For example, a warning can be issued to the driver of the vehicle after or during step 350.

In particular, it can be checked in step 340 whether the gas component is also present outside the battery housing 110 at the same time. If this is the case, it can be assumed that the gas component has flowed into the battery housing 110 from outside same and consequently the first gas sensor 210 also detects this gas component and consequently the first gas signal indicates the presence and/or a content of the gas component within the battery housing 110 that exceeds the predetermined first content threshold value.

If, on the other hand, it is determined in step 340 that no gas component has (yet) been detected or only a small amount of the gas component has been detected outside the battery housing 110, it can be assumed that the gas component that has been detected by the first gas sensor 210 inside the battery housing 110 has also been generated inside the battery housing 110 and consequently there is most likely a defect in the battery arrangement 100, for example the at least one battery cell 120, and this can be detected accordingly.

Furthermore, the teachings herein may provide a redundant gas measurement for the battery arrangement 100 by means of the two gas sensors 210, 220. For example, the time sequence in which the first gas sensor 210 and the second gas sensor 220 detect the gas component can be analyzed. If the second gas sensor 220 detects the gas component before the first gas sensor 210, it can be assumed that the gas mixture including the gas component is flowing or has flowed into the battery housing 110 from the outside. However, if the first gas sensor 210 detects the gas component before the second gas sensor 220, it can be assumed that the gas mixture including the gas component flows or has flowed out of the interior of the battery housing 110.

If only one of the two gas sensors 210, 220 detects the gas component within a predetermined period of time, it can be assumed that one of the two gas sensors 210, 220 is defective. In particular, said predetermined period of time is such that, if the two gas sensors 210, 220 are functioning properly, both gas sensors 210, 220 should also detect the gas components, in particular due to the presence of the pressure equalization device 250.

In addition, the device 200 can be designed to recognize the case in which the first gas sensor 210 detects the gas component much later than the second gas sensor 220. Rather, in a case in which the gas component flows into the interior of the battery housing 110 from the outside, the first gas sensor 210 should have detected the gas component a relatively short time after the second gas sensor 220 has detected the gas component. However, if the first gas sensor 210 only detects the gas component after a predetermined period of time has elapsed, such as in the range of 1 to 2 minutes, after the second gas sensor 220 has detected the gas component, it can be concluded that the battery arrangement 100 is defective. In this case, the origin of the gas component detected by the first gas sensor 210 is probably inside the battery housing 110 and it is not a gas component that has flowed into the interior of the battery housing 110 from outside.

What is claimed is:

1. A device for detecting a defect of a battery arrangement including a battery housing and at least one battery cell arranged in the battery housing, the device comprising:
a first gas sensor sensitive to at least one gas component if present within the battery housing and operating to generate a first gas signal indicating presence and/or a content of the at least one gas component within the battery housing;
a second gas sensor sensitive to the at least one gas component if present outside the battery housing and operating to generate a second gas signal indicating presence and/or a content of the at least one gas component outside the battery housing; and
a control apparatus connected to the first gas sensor and the second gas sensor and receiving the first gas signal and the second gas signal;
wherein the control apparatus identifies a defect in the battery arrangement if the first gas signal indicates the presence and/or a content of the gas component within the battery housing exceeding a predetermined first content threshold value and the second gas signal indicates the absence and/or a content of the gas component outside the battery housing falling below a predetermined second content threshold value.

2. The device as claimed in claim 1, further comprising
a device housing;
wherein the first gas sensor and the second gas sensor are at least partially arranged in the device housing; and
the device housing designed to be attached to the battery housing so an opening in the battery housing is at least partially sealed airtight by the device housing.

3. The device as claimed in claim 2, further comprising
a printed circuit board arranged in the device housing;
wherein the first gas sensor and the second gas sensor are electrically connected to the printed circuit board; and
the printed circuit board electrically connected to the control device.

4. The device as claimed in claim 2, wherein:
the first gas sensor extends sealingly from an interior of the device housing at least partially into an interior of the battery housing; and/or
the second gas sensor extends sealingly from the interior of the device housing into an environment surrounding the device housing and the battery housing.

5. The device as claimed in claim 2, further comprising
a pressure equalization device operable to at least partially equalize a pressure difference between an inside of the battery housing and an outside of the battery housing and to aerate or deaerate the battery housing.

6. The device as claimed in claim 5, wherein the pressure equalization device is arranged on the device housing so any fluid flowing out of the battery housing or flowing into the battery housing through the pressure equalization device flows at least partially past the second gas sensor.

7. The device as claimed in claim 5, further comprising
an emergency deaeration device to deaerate the battery housing when the pressure inside the battery housing exceeds a predetermined pressure threshold value.

8. The device as claimed in claim 7, wherein the emergency deaeration device is arranged on the device housing so any fluid flowing out of the battery housing through the emergency deaeration device flows at least partially past the second gas sensor.

9. The device as claimed in claim 5, further comprising
a flow guiding attached to the device housing and extending at least partially into the interior of the battery housing so the fluid flowing out of the battery housing through the pressure equalization device is at least partially guided past the first gas sensor.

10. The device as claimed in claim 2, further comprising
a device flange attached to the device housing and by means of which the device housing can be fastened to the battery housing.

11. The device as claimed in claim 2, wherein the device housing includes:
a first housing region fluidically connected to an interior of the battery housing; and
a second housing region fluidically connected to an exterior of the battery housing and separated from the first housing region by means of a housing wall.

12. A battery arrangement comprising:
battery housing;
at least one battery cell arranged in the battery housing;
a first gas sensor sensitive to at least one gas component if present within the battery housing and operating to generate a first gas signal indicating presence and/or a content of the at least one gas component the battery housing;
a second gas sensor sensitive to the at least one gas component if present outside the battery housing and operating to generate a second gas signal indicating presence and/or a content of the at least one gas component outside the battery housing; and
a control apparatus connected to the first gas sensor and the second gas sensor and receiving the first gas signal and the second gas signal;
wherein the control apparatus identifies a defect in the battery arrangement if the first gas signal indicates the presence and/or a content of the gas component within the battery housing exceeding a predetermined first content threshold value and the second gas signal indicates the absence and/or a content of the gas component outside the battery housing falling below a predetermined second content threshold value.

13. A method for detecting a defect of a battery arrangement having a battery housing and at least one battery cell arranged in the battery housing, the method comprising:
generating a first gas signal using a first gas sensor sensitive to at least one gas component if present inside the battery housing;
generating a second gas signal using a second gas sensor sensitive to the at least on gas component if present outside the battery housing; and
identifying a defect in the battery arrangement if the first gas signal indicates the presence and/or a content of the gas component inside the battery housing exceeding a predetermined first content threshold value, and the second gas signal indicates the absence and/or a content of the gas component outside the battery housing falling below a predetermined second content threshold value.

14. The method as claimed in claim 13, wherein the second gas signal is generated after a predetermined period of time has elapsed after the first gas signal has been generated; and
the method further comprises identifying
a defect in the battery arrangement if the first gas signal indicates the presence and/or a content of the gas component inside the battery housing exceeding a predetermined first content threshold value, and the second gas signal indicates the presence and/or a content of the gas component outside the battery housing falling below a predetermined third content threshold value.

15. The method as claimed in claim 13, further comprising
identifying a defect of the first gas sensor and/or the second gas sensor if only one of the two gas sensors generates a gas signal indicating the presence and/or a content of the gas component which is greater than the predetermined first content threshold value.
